(19) United States
(12) Patent Application Publication
(10) Pub. No.: US 2005/0267429 A1
(43) Pub. Date: Dec. 1, 2005

(54) Acquisition/distribution layer

(76) Inventor: Richmond R. Cohen, Williamsport, PA (US)

Correspondence Address: AMSTER, ROTHSTEIN & EBENSTEIN LLP, 90 PARK AVENUE, NEW YORK, NY, 10016, US

(21) Appl. No.: 10/852,803

(22) Filed: May 25, 2004

(51) Int. Cl. A61F 13/15

(52) U.S. Cl. 604/378

(57) ABSTRACT

An absorbent article has a topsheet, an absorbent core and an acquisition/distribution transfer system disposed intermediate the topsheet and the absorbent core. The system includes at least one apertured material which is three dimensional and defines pores extending appreciably beyond the primary plane of the material in a direction from the absorbent core toward the topsheet. Preferably the pores taper inwardly from the core toward the topsheet.

### BACKGROUND OF THE INVENTION

The present invention relates to disposable absorbent articles such as diapers, and more particularly to a novel acquisition/distribution layer therefor, interposed between the topsheet and the absorbent care.

Disposable absorbent articles such as baby diapers, adult diapers, and feminine hygiene products today typically have multiple layers of absorbent materials or composites. The articles always have a topsheet and an absorbent core. The absorbent core is generally a composite of cellulosic fluff pulp and superabsorbent polymer (SAP) that stores most of the liquid entering the article through the topsheet. Most diapers also contain an acquisition/distribution layer (ADL) interposed between the topsheet and the absorbent core. The functions of the ADL include improvement of the rate of liquid uptake into the diaper (i.e., increase the liquid acquisition speed), improvement in the retention of liquid in the diaper (i.e., lower the rewet or wetback characteristics), and improvement in spreading the liquid throughout the diaper to utilize its capacity more effectively (i.e., the distribution or wicking factor which affects both the acquisition rate and rewet characteristics).

The material interposed between the topsheet and the absorbent core ideally acts as an acquisition/distribution layer which receives the liquid of a liquid insult from the topsheet, provides additional capacity for the liquid, and distributes it laterally before it enters the absorbent core. This distribution of liquid prevents over-saturation of a local area of the absorbent core by increasing the surface area of the core receiving the liquid and providing more time for the core to accept the liquid. Being well-distributed, the liquid from the ADL is better absorbed by the absorbent core because it avoids the formation of liquid pools in an over-saturated local area of the absorbent core. Thus the acquisition/distribution layer not only improves strike-through (that is, the time required to absorb the liquid insult) but also improves rewet characteristics (that is, the amount of liquid which leaks back from the absorbent core through the acquisition/distribution layer under pressure).

The importance of the acquisition/distribution layer becomes more evident with subsequent liquid insults directed to the same local area of the core as the local area tends to already be filled with liquid from the previous liquid insult. In the absence of an effective ADL, the difficulty in wicking or distribution of the initial liquid insult leaves the local area of the core already wet and thus less capable of handling subsequent liquid insults.

As discreetness is an important issue for many wearers of absorbent products, diapers that are termed “thin” are becoming more prevalent. Generally, these diapers are rendered thin by replacing a significant percentage of the fluff pulp with SAP and then compressing the absorbent core. Although such techniques are effective in providing a thinner diaper, the absorbent properties of the diaper may be compromised. With the combination of compression and increased SAP content, thin diapers tend to show slow speeds of liquid acquisition and reduced wicking and spreading of liquid. As a result, such structures are more prone to leakage. Such is the case regardless of the properties of the SAP. Hence, the enhancement in discreetness, comfort and fit developed by making a thin structure may be offset by poor absorbency.

One means of increasing liquid intake and decreasing leakage in thin absorbent products is through improvement to the ADL. Most acquisition-layer materials are nonwoven materials. They are typically fabrics comprised of thermoplastic fibers such as polyester or polyolefins that are often thermobonded, through-air bonded, or resin bonded. Some ADL are cellulose-based or combine cellulosic fibers with thermoplastic fibers. One means of improving such materials for thin cores is to increase their basis weight in the structure. Although such a tactic provides some level of improved absorbent performance, it hurts the economics of manufacturing the product.

Gaining in usage lately are ADLs that are apertured polyethylene materials. Such films are rendered hydrophilic with a durable surfactant. The most effective ADL of this type is a three-dimensional film, i.e., a film that has apertures formed as extended conical pores which taper or decrease in diameter with distance from the primary plane of the film. The orientation of such a film in an absorbent product is with the projecting cones (i.e., the truncated apices of the cones) facing the absorbent core and the smooth side of the film (i.e., the bases of the cones) facing the topsheet. The rationale for this orientation is that the tapering cones will provide superior drainage of the liquid toward the core and inhibit rewetting back through the topsheet. See, for example, U.S. Pat. No. 4,324,247. Furthermore, the smooth side of the film would be expected to provide better aesthetics (e.g., hand) than the rougher side with the projecting cones. In absorbent products, such ADL materials in thin core structures tend to reduce rewetting relative to fiber-based acquisition materials (such as nonwoven ADLs) but do not provide significantly large improvements in acquisition speeds relative to higher-loft nonwoven ADLs. Such improvements in acquisition speeds may only occur if the permeability of the apertured films is increased as the technology for making such improvements evolves. Such increases in permeability are achieved, for example, by creating larger apertures. However, the larger apertures may also result in higher rewets.

It is the acquisition speed that is in most critical need of improvement for a thin core diaper structure. Thus, what is needed is an ADL that can perform better in conjunction with a thin absorbent core. Such a structure could be an ADL that is not necessarily heavier, but includes a novel design that enables it to have a special synergy with the thin absorbent core—that is, it is designed to improve simultaneously the ability of the core to absorb faster, retain liquid better and enhance the spreading and wicking of liquid.

Accordingly, it is an object of the present invention to provide an absorbent structure which, in a preferred embodiment, improves simultaneously the ability of the core to absorb faster, retain liquid better and enhance the spreading and wicking of liquid.

Another objective provides such an absorbent structure wherein, in a preferred embodiment, the ADL has a special synergy with the thin absorbent core.

A further objective provides such an absorbent structure which, in a preferred embodiment, is simple and inexpensive to manufacture and use.

### SUMMARY OF THE INVENTION

It has now been found that the above and related objects of the present invention are obtained in an absorbent article comprising a topsheet, an absorbent core, and an acquisition/distribution transfer system disposed intermediate the topsheet and the absorbent core. The system comprises at least one apertured material, the one apertured material being three dimensional and defining pores extending appreciably beyond the primary plane of the material in a direction from the absorbent core toward the topsheet. Preferably the pores taper inwardly in a direction from the absorbent core toward the topsheet.

In a preferred embodiment where the system consists essentially of only one apertured material, the only one apertured material is three dimensional and defines pores which extend appreciably beyond the primary plane of the material in a direction from the absorbent core toward the topsheet and preferably taper inwardly in a direction from the absorbent core toward the topsheet.

The material has an average pore size of 0.3-10, preferably 0.5-5.0, and optimally 1.0-2.0 mm in diameter, and a basis weight of 25-100, preferably 30-65, and optimally 35-50 gsm. Preferably the pores are generally conical, and the material is formed of a wettable and substantially non-absorbent thermoplastic polymer, such as polyethylene.

In a preferred embodiment where the system consists essentially of at least a pair of apertured materials, including a first material facing the topsheet and a second material facing the absorbent core, each material is three dimensional. At least the first material defines pores which extend appreciably beyond the primary plane of the material in the first direction and preferably taper inwardly in a first direction from the absorbent core to the topsheet, the first material having a larger average pore size than the second material.

The materials have a combined thickness of at least 30 mils (0.76 mm), optimally at least 50 mils (1.3 mm). The first material has an average pore size of 0.3-10, preferably 0.5-5.0, and optimally 1.0-2.0 mm in diameter, and the second material has an average pore size of 0.1-2.0, preferably 0.3-1.5, and optimally 0.5-1.0 mm in diameter.

The first material has a basis weight at least as high as the second material. The first material has a basis weight of 25-100, preferably 30-65, and optimally 35-50 gsm, and the second material has a basis weight of 10-35, preferably 15-30, and optimally 20-30 gsm.

Preferably, the first and second materials are contiguous and may be laminated together. Each of the materials is preferably formed of a wettable and substantially non-absorbent thermoplastic polymer, preferably substantially the same polymer (such as polyethylene). The pores are conical.

### BRIEF DESCRIPTION OF THE DRAWING

The above and related objects, features and advantages of the present invention will be more fully understood by reference to the following detailed description of the presently preferred, albeit illustrative, embodiments when taken in conjunction with the accompanying drawing wherein:

FIG. 1 is a sectional view of an absorbent structure according to the present invention having a single-layer ADL; and

FIG. 2 is a view similar to FIG. 1 of an absorbent structure having a multi-layer ADL.

### DETAILED DESCRIPTION OF THE PREFERRED EMBODIMENTS

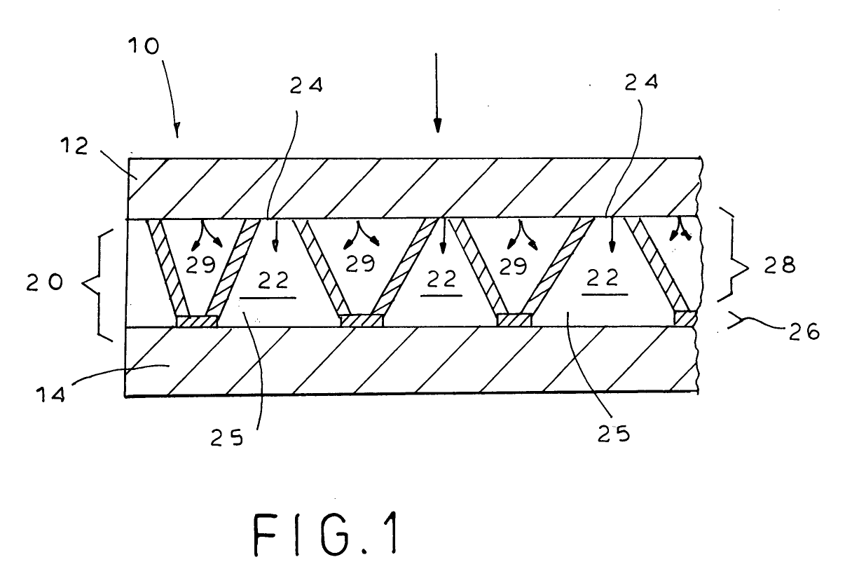

Referring now to the drawing, and in particular FIG. 1 thereof, therein illustrated is an absorbent structure according to the present invention, generally designated by the reference numeral 10, such as a diaper, incontinence pad, sanitary napkin or the like.

The absorbent structure 10 comprises a topsheet or coversheet 12, an absorbent core 14, and an ADL 20 therebetween. The ADL 20 comprises in turn a hydrophilic, flexible three-dimensional apertured material whose orientation in the absorbent structure 10 is such that its inwardly tapering hollow pores 22 are facing toward the topsheet 12 and its smooth side is facing toward the absorbent core 14. Such an orientation, which is the opposite of what conventional wisdom and practice would dictate (see U.S. Pat. No. 4,324,247), delivers the surprising results of superior absorbent performance versus the standard orientation in which the inwardly tapering hollow pores 22 face toward the absorbent core 14. With the orientation of the ADL according to the present invention, the acquisition speed in the absorbent article 10 is greatly enhanced for thin diaper cores without sacrificing either rewet properties or aesthetics.

The apertured material 20 illustrated in the drawing is a thermoplastic polymeric film, such as a polyolefin (e.g., polyethylene, polypropylene or the like), with pore openings sufficiently large to enable rapid liquid acquisition. Where the thermoplastics that comprise such materials are naturally hydrophobic, they must be treated with a wetting agent or agents and would otherwise be unsuitable without such treatment in an absorbent structure. These wetting agents may be topically applied to the material or may be present therein in the form of an internal additive. It is important that the wetting system impart durable hydrophilicity to the material so that the material is able to maintain hydrophilicity despite repeated “insults.” That is, it is essential that not all of the wetting agent is washed off during the first insult.

In a preferred embodiment of the present invention, the ADL 20 comprises a thin, flexible material with conical pore openings 22 tapering inwardly, preferably a substantially non-absorbent polyethylene film. The truncated apices 24 of the pores 22 project from the main or primary plane 26 of the material 20 towards the topsheet 12, and the bases 25 of the pores 22 are within the main or primary plane 26 of the material 20. The material 20 has an average pore size of about 0.3-10 mm in diameter, preferably about 0.5-5.0 mm in diameter, optimally about 1.0-2.0 mm in diameter. The material 20 contains a sufficiently high concentration of wetting agent so as to be durably wettable (i.e. hydrophilic).

The basis weight of the apertured material is about 25-100 gsm, preferably about 30-65 gsm, and optimally about 35-50 gsm. The thickness of the apertured material is about 10-150 mils, preferably about 15-75 mils, and optimally about 20-60 mils. Thickness is measured by a Digital Micrometer, Model 49-72, available from Testing Machines, Inc. (with a 2 inch diameter anvil for applying a load of 95 g/in² to the sample). Preferably the acquisition/distribution layer has a density not exceeding 0.07 g/cc so as to provide sufficient loft or thickness to the system.

The pores 22 of the material 20 may be formed by conventional means well-known in the art. One preferred technique for a film involves the use of heat and suction. Thus each generally planar material is heated to its softening point (below the melting point), and then suction is applied to one side of the material to form the pores 22. The suction draws a portion of the material outwardly, typically through an apertured screen, so that pores 22 of the desired configuration are formed within the material. In this preferred technique of forming the pores 22, at least a portion of the material drawn out of the main plane 26 of the material 20 by the suction remains a part of the material and projects outwardly from the main plane 26 of the material as hollow projections 28.

The projections 28 are on average at least 40, preferably at least 50-100, times greater in thickness than the main plane 26 of the material 20 and thus preferably provide about 95% of the total loft of the material, the remaining 5% being provided by the main plane 26 of the material 20. The material thickness measurements provided hereinbelow and in the Examples include the projections 28.

Upon subsequent assembly of the absorbent article 10, the tops or truncated apices of the upward projections 28 of a film 20 contact and locally space the topsheet 12 above the main plane 26 of the film 20 by the thickness of projections 28. The presence of the projections 28 desirably increases the overall loft or thickness of the ADL and, in particular, creates laterally extending channels 29 intermediate the bottom of the topsheet 12 and the top of the main plane 26 of the film 20 so that liquid from a liquid insult (see the large single-headed arrow) can easily pass laterally between the topsheet 12 and the primary plane 26 (see the small double-headed arrows). Thus, liquid which passes through the topsheet 12 and emerges therefrom to find no pore 22 of the film 20 directly therebelow (that is, no small single-headed arrow), is able to travel laterally through such channels 29 until it finds an adjacent pore 22 of the film 20 into which it can enter, thereby providing additional capacity and additional time for the absorbent core 14 to absorb the liquid and avoiding a local bulking of the liquid.

It will be appreciated that the projections 28 are formed by relatively thin walls, which preferably, but not necessarily, continue the taper of pores 22. The thin walls form only loose contacts with the topsheet surface above so that the liquid can pass through the loose contacts and enter into the laterally extending channels between the topsheet 12 and the main plane 26 of the material 20.

While it is preferred that the pores 22 and projections 28 cooperatively define the configuration of truncated cones, the benefits of the present invention can be obtained, albeit perhaps to a lesser degree, where the pores 22 and projections 28 cooperatively form a different, non-conical configuration, and even where the pores and projections do not taper from the core 14 towards the topsheet 12.

While the material 20 useful as the ADL includes a three dimensional apertured film, apertured nonwoven, or other permeable structures known to those skilled in the absorbent article art (e.g., apertured hydrophilic foam), the three dimensional apertured films are preferred, especially a three dimensional apertured polyethylene film with conical pores, characterized by a thickness of 50 mil and a basis weight of 36 gsm, available under the trade name AQUIDRY from Tredegar Film Products.

To understand why the orientation of an ADL according to the present invention has such a large impact on the acquisition speeds, it is necessary to consider the physics of the liquid flow. First, although there is a small impact from capillarity when an insulting liquid strikes an absorbent article, the dominant pressure that drives flow in most cases is created by the kinetic energy of the oncoming liquid. Hence, we will neglect capillary pressure in this analysis. If we consider the pores of the ADL as gradually contracting or expanding cylindrical pipes, fluid mechanics teaches that steady-state flow through such pipes is essentially the same whether we consider a gradual contraction or a gradual expansion. Hence, flow through the pores of the ADL itself is not sufficient to explain the phenomenon of the invention, and we need to analyze the ADL as part of the layered absorbent structure.

Consider the three-layered structure of a topsheet, ADL and core in an absorbent article. The core, composed of a mixture of compressed fluff pulp and SAP, will have the lowest average pore size and hence the lowest permeability to liquid flow. The topsheet has an average pore diameter that is much larger than the core, and, if appropriately treated with a surfactant to render it hydrophilic, will allow penetration of liquid more rapidly than the core. Thus, the rate-limiting step in the liquid-transfer process is movement of liquid into the core. To give the core more time to absorb the liquid, it is important for the ADL to provide space or additional capacity while the liquid enters through the topsheet. The three-dimensional apertured material in the “reverse” orientation of the present invention accomplishes this.

More particularly, it is hypothesized that the loft created by the truncated apices of the projecting cones of the apertured material creates space between two highly permeable materials, that is, the topsheet and the ADL. This space manages the excess liquid that is entering more rapidly through the topsheet than the core, thereby allowing time for the lower permeability core to accept the liquid. If the ADL is in the conventional orientation, there is space between the core and the ADL. However, the projecting apertures are conducting the flow, and the openings at the truncated apical ends of the cones are in direct contact with the core, so the space between the ADL and the core is not as readily useable to manage excess liquid as in the case when the ADL orientation is reversed so that the space is between the ADL and the topsheet.

The physics describing liquid flow in rewet measurements is different than it is for acquisition-speed testing. During rewet, the application of a weight provides the impetus for liquid flow. Capillary pressure plays a more significant role in driving or inhibiting liquid flow.

For this reason, considering the same three-layered configuration described above, it would be logical to think that the tapering pores of the three-dimensional ADL material would create a capillary-pressure gradient that in the CSD configuration (cone side down facing the absorbent core) would inhibit rewet and in the CSU configuration (cone side up facing the topsheet) would promote higher rewet. However, this expected effect is not manifested.

It is hypothesized that the failure of the orientation to produce a significant impact on the rewet is explained by the fact that the capillary pressure for the ADL is low at both ends of these large pores, so that the capillary-pressure gradient from one end to the other is not meaningful in affecting liquid flow.

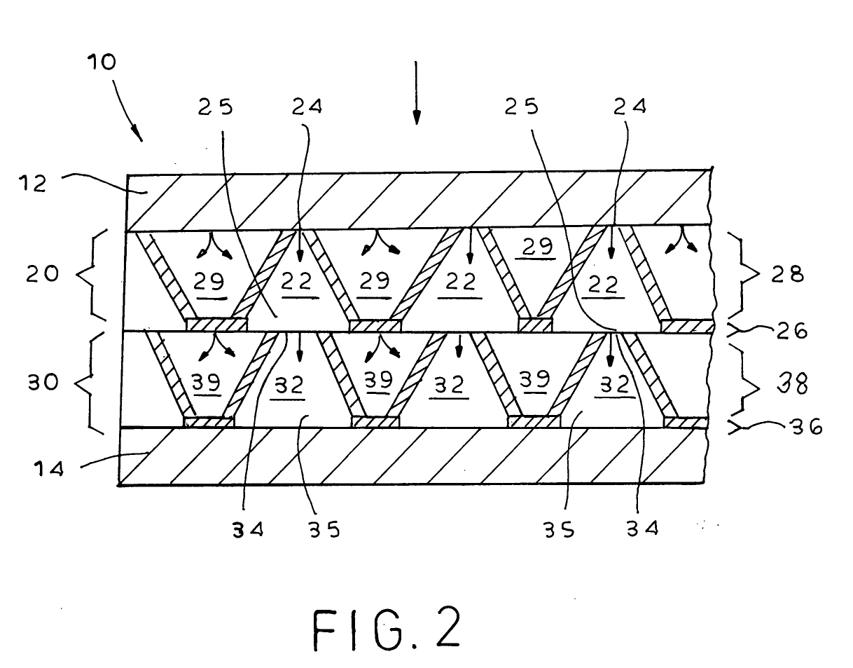

Referring now to FIG. 2 in particular, in an alternative embodiment of the absorbent structure 10′, the apertured material 20 may be used in conjunction with additional 3D layers of apertured or non-apertured nonwoven, apertured material or other permeable structures know to those skilled in the absorbent article art (e.g., apertured hydrophilic foam) These additional materials may be arranged to be between the topsheet 12 and the apertured material 20 of the present invention or, as illustrated, between the apertured material 20 of the present invention and the absorbent core 14. In either case, the orientation of the apertured material 20 is such that the truncated apices 24 of the cones 22 face the topsheet 12 and the bases 25 of the cones 22 are in the main or primary plane of the material 26 facing the absorbent core 14.

Thus, the acquisition/distribution system may consist essentially of at least a pair of three dimensional apertured materials 20, 30 (including a first material 20 facing the topsheet 12 and a second material 30 facing the absorbent core 14). At least the first material 20 (shown as an apertured film) has pores 22, which taper inwardly in a direction from the absorbent core 14 to the topsheet 12, the pores 22 forming projections 28 (extending appreciably beyond the primary plane 26 of the film 20 in the same direction) and channels 29. The second material 30 (also shown as an apertured film) preferably is of the same general configuration, with pores 32 , truncated apices 34, bases 35, a primary plane 36 of film 30, projections 38 and channels 39.

In this instance, the two materials 20, 30 have a combined thickness of at least 30 mils (0.76 mm) and preferably at least 50 mils (1.3 mm). Typically, although not necessarily, the first material 20 has a larger average pore size than the second material 30, the first material 20 having an average pore size of 0.3-10 mm in diameter, preferably 0.5-5.0 mm, and optimally 1.0-2.0 mm, and the second material 30 having an average pore size of 0.1-2.0 mm in diameter, preferably 0.3-1.4 mm, and optimally 0.5-1.0 mm. Preferably the first material 20 has a basis weight of at least as high as the second material 30, the first material having a basis weight of 25-100 gsm, preferably 30-65 psm and optimally 35-50 gsm, and the second material having a basis weight of 10-35 gsm, preferably 15-30 gsm, and optimally 20-30 gsm.

The first and second materials 20, 30 are preferably laminated or bonded together about the lateral periphery of the materials, so that the first and second materials are contiguous. Preferably both materials are formed of substantially the same polymer (preferably polyethylene) and have pores which are generally conical.

Preferred second materials 30 are three dimensional polyethylene films characterized by smaller cones than the AQUIDRY material, available under the trade name 25475 from Tredegar. Also useful as the second materials 30 are nonwovens such as the durable finish 15 gsm polypropylene spunbond nonwoven available from First Quality Nonwovens, and the 50 gsm resin bonded polyester nonwoven available under the trade name 9342736 from BBA Nonwovens.

It should be appreciated that the nonwovens used as the additional material 30 need not have apertures formed therein, with reliance being placed on the naturally formed pores or interstices of the nonwoven for liquid permeability. On the other hand, the nonwovens used as the basic material 20 must be intentionally apertured post-production in order to provide the desired projections 28 in the nonwoven. Of course, the apertured nonwovens may also be used for the material 30. Where the nonwoven includes both naturally formed interstices and post-production formed apertures, only the latter are included in determining the average pore size of the material.

The unique orientation of the apertured material according to the present invention, when used in the body of an absorbent article 10, yields faster acquisition speeds than when the material is oriented in the conventional direction. The benefits of the invention are particularly evident with a thin absorbent core 14. The aesthetic differences of the apertured material 20, in its novel orientation versus its conventional orientation, are difficult to detect when a suitable topsheet 12 is placed above the ADL 20 in an actual absorbent article 10.

The efficacy of the present invention was measured using the following test procedures.

Test Methods

The test procedure used to evaluate the performance of the invention measures the acquisition time and rewet of an absorbent structure for multiple insults. The procedure is similar to others that are widely used in the field.

The absorbent structure is laid flat on a surface; leg gathers are trimmed, if applicable, to accomplish this. A dosing ring (60 mm I.D., 70 mm O.D., and 40 mm height) is placed on the targeted areas of the absorbent structure.

Then, 100 ml of synthetic urine (0.9% NaCl solution) is measured in a graduated cylinder and poured into a 125 ml separatory funnel. The funnel discharges liquid at a rate of 9 ml/s when its stopcock valve is opened fully. Positioning the bottom tip of the funnel 40 mm from the surface of the absorbent structure in the center of the dosing ring, the stopcock is fully opened, and the synthetic urine is dispensed onto the absorbent structure. Simultaneously, a timer is activated. The timer is stopped when the 100-ml dose completely passes through the topsheet. This time is recorded as the first acquisition time.

The dosing ring is now removed and another timer is activated to measure 15 minutes. After 15 minutes, a stack of pre-weighed filter paper (AFI Grade 950, 9 cm diameter) weighing about 10 g is placed in the center of the wetted target area. A cylindrical weight applying 1 psi of pressure is placed on top of the filter paper, with the weight having a diameter also of 9 cm. After waiting 1 minute, the weight is removed, and the filter paper is weighed. The difference in weight is recorded as the first rewet.

Two additional 100-ml doses of synthetic urine are applied using almost the identical procedure outlined above to produce a total of three “insults” per absorbent structure. For the second and third insults, 15 g of filter paper is used.

The total number of replicates is either 5 or 10 per absorbent structure. The average values of the acquisition times and rewets plus the standard deviations are computed.

### EXAMPLES

### Example 1

Absorbent structures were prepared comprising in sequence:

(i) a 13.gsm liquid-permeable nonwoven topsheet of polypropylene spunbond nonwoven (0.150 mm thick) available under the trade name SB1350021 from First Quality Nonwovens,

(ii) an ADL (see below for details),

(iii) a 300 gsm thin absorbent core of cellulose fluff and SAP (about 50:50 ratio), laminated with tissue on the back, available under the trade name NOVATHIN from Rayonier, Inc., and

(iv) a liquid-impermeable film backsheet of polyethylene (1.1 mm thick) available under the trade name DH-203 from Clopay Plastic Products.

The absorbent core and topsheet were cut to 21″ long and 4.25″ wide. The ADL was cut to 21″ long and 3.25″ wide.

The ADL was selected from a group consisting of:

AD: a 3D (50 mil thick) apertured polyethylene film of 36 gsm with conical pores, available under the trade name AQUIDRY from Tredegar Film Products,

DW: a 3D (about 14 mil thick) apertured polyethylene film of about 25 gsm with conical pores, available under the trade name 25475 from Tredegar Film Products,

DFPPSB: a durable finish 15 gsm polypropylene spunbond available from First Quality Nonwovens,

NW: a 50 gsm resin bonded polyester nonwoven available under the trade name 9342736 from BBA Nonwovens, and combinations thereof.

The ADL was oriented either with the truncated apices of the cones facing the topsheet (CSU) or the truncated apices of the cones facing the absorbent core (CSD).

The composition and orientation of each absorbent structure and its performance data are recorded in Table 1.

Generally Table 1 shows great improvement in acquisition speeds when the films with the larger pores (AD) are in the CSU orientation.

From the data of Samples 1 and 2, it is evident that the absorbent structure that showed the lowest acquisition times occurred when the CSU orientation of the present invention was deployed (rather than the conventional CSD orientation). The differences in the rewets between the CSU and CSD orientations are not statistically significant.

A comparison of the data of Samples 9 and 10 and for Samples 11 and 12 confirm the findings from the comparison of Samples 1 and 2—namely, that the absorbent structure that showed the lowest acquisition times occurred when the CSU orientation of the present invention was deployed. (The marginally higher second rewet time for Sample 10 is believed to be an experimental anomaly.) A comparison of the data of Samples 7 and 8 shows that when multi-layer ADLs are used, the lowest acquisition times occur when both layers are in the CSU orientation of the present invention, rather than one in the CSU orientation and one in the CSD orientation. This comparison further shows that the enhancement in acquisition time is achieved without any appreciable penalty in rewet.

The data for Samples 3 through 6 confirm that excellent acquisition times are achieved regardless of the order of the large cone AD or small cone DW layers (that is, regardless of which is closer to the topsheet).

A comparison of the data for Samples 5 and 8 shows that while the faster acquisition times are achieved regardless of whether the large cone AD or small cone DW layer is closest to the topsheet, better rewets are obtained with the large cone AD closer to the topsheet.

### Example 2

Absorbent undergarment products were produced on a diaper machine and comprised

(i) a 13.5 gsm nonwoven topsheet (as in Example 1);

(ii) an ADL consisting of a 3D (50 mil thick) apertured polyethylene film of 36 gsm with conical pores, available under the trade name AQUIDRY from Tredegar Film Products (as in Example 1),

(iii) a 550 gsm absorbent core containing fluff pulp and SAP, and

(iv) an impermeable film backsheet (as in Example 1).

The ADL was included in the undergarments in either the CSU or CSD orientations. The undergarments containing the ADL in the CSD orientation were the large protective underwear product available under the trade name PV-513 from First Quality Products, and the other undergarments were similar except for the CSU orientation of the ADL.

Volunteers wore the undergarments overnight. Five of the 10 volunteers (2 male and 8 female) were unable to distinguish any comfort difference between the products.

To summarize, the present invention provides an absorbent structure which improves simultaneously the ability of the core to absorb faster, retain liquid better and enhance the spreading and wicking of liquid. The ADL has a special synergy with a thin absorbent core and is simple and inexpensive to manufacture and use.

Now that the present invention has been described in detail, various modifications and improvements thereon will become readily apparent to those skilled in the art. Accordingly, the spirit and scope of the present invention is to be construed broadly and limited only by the appended claims, and not by the foregoing specification.

TABLE 1

| | | 1^{st} | 2^{nd} | 3^{rd} | 1^{st} | 2^{nd} | 3^{rd} |
|---|---|---|---|---|---|---|---|
| Sample | ADL | Time | Time | Time | Rewet | Rewet | Rewet |
| # | Orientation | (s) | (s) | (s) | (g) | (g) | (g) |
| 1 | AD-CSD | 14.6 | 17.8 | 24.4 | .09 | 5.39 | 9.86 |
| | (Control) | | | | | | |
| 2 | AD-CSU | 12.0 | 12.2 | 12.8 | .09 | 5.86 | 8.61 |
| 3 | AD-CSU | 12.4 | 12.0 | 12.6 | .09 | 4.36 | 7.71 |
| | DW-CSD | | | | | | |
| 4 | AD-CSD | 12.8 | 12.4 | 13.6 | .08 | .63 | 2.52 |
| | DW-CSU | | | | | | |
| 5 | AD-CSU | 12.0 | 12.2 | 12.0 | .10 | 2.71 | 4.16 |
| | DW-CSU | | | | | | |
| 6 | DW-CSD | 12.0 | 12.0 | 13.4 | .12 | 4.41 | 7.27 |
| | AD-CSU | | | | | | |
| 7 | DW-CSU | 13.0 | 14.4 | 18.8 | .10 | 5.28 | 8.21 |
| | AD-CSD | | | | | | |
| 8 | DW-CSU | 12.0 | 12.0 | 12.6 | .23 | 5.12 | 7.11 |
| | AD-CSU | | | | | | |
| 9 | DFPPSB | 14.4 | 17.2 | 23.8 | .32 | 4.83 | 9.08 |
| | AD-CSD | | | | | | |
| | (Control) | | | | | | |
| 10 | DFPPSB | 13.0 | 12.2 | 13.2 | .36 | 6.96 | 9.20 |
| | AD-CSU | | | | | | |
| 11 | NW | 14.6 | 18.4 | 27.8 | .19 | 6.36 | 9.30 |
| | AD-CSD | | | | | | |
| | (Control) | | | | | | |
| 12 | NW | 12.4 | 12.0 | 12.6 | .27 | 6.20 | 9.18 |
| | AD-CSU | | | | | | |

1. An absorbent article comprising:
(A) a topsheet;
(B) an absorbent core; and
(C) an acquisition/distribution transfer system disposed intermediate said topsheet and said absorbent core;
said system comprising at least one apertured material, said one apertured material being three dimensional and defining pores extending appreciably beyond the primary plane of the material in a direction from said absorbent core toward said topsheet.

2. The absorbent article of claim 1 wherein said pores taper inwardly in a direction from said absorbent core toward said topsheet.

3. The absorbent article of claim 1 wherein said system consists essentially of one of
(i) only one apertured material, and
(ii) at least a pair of apertured materials including a first material facing said topsheet and a second material facing said absorbent core;
each said only one material, said first material and said second material being three dimensional and defining pores which taper inwardly in a direction from said absorbent core toward said topsheet and extend appreciably beyond the primary plane of the material in a direction from said absorbent core toward said topsheet, said first material having a larger average pore size than said second material.

4. The absorbent article of claim 1 wherein said system consists essentially of said only one apertured material, said only one apertured material being three dimensional and defining pores which taper inwardly in a direction from said absorbent core toward said topsheet and extend appreciably beyond the primary plane of the material in a direction from said absorbent core toward said topsheet.

5. The article of claim 4 wherein said material has an average pore size of 0.3-10 mm in diameter.

6. The article of claim 5 wherein said material has an average pore size of 0.5-5.0 mm in diameter.

7. The article of claim 6 wherein said material has an average pore size of 1.0-2.0 mm in diameter.

8. The article of claim 4 wherein said material has a basis weight of 25-100 gsm.

9. The article of claim 8 wherein said material has a basis weight of 30-65 gsm.

10. The article of claim 9 wherein said material has a basis weight of 35-50 gsm.

11. The article of claim 4 wherein said material is formed of polyethylene.

12. The article of claim 4 wherein said pores are generally conical.

13. The article of claim 4 wherein said material is formed of a wettable and substantially non-absorbent thermoplastic polymer.

14. The absorbent article of claim 3 wherein said system consists essentially of at least a pair of apertured materials including a first material facing said topsheet and a second material facing said absorbent core, each said material being three dimensional, at least said first material defining pores which taper inwardly in a first direction from said absorbent core to said topsheet and extend appreciably beyond the primary plane of the material in said first direction, said first material having a larger average pore size than said second material.

15. The article of claim 14 wherein said materials have a combined thickness of at least 30 mils (0.76 mm).

16. The article of claim 14 wherein said materials have a combined thickness of at least 50 mils (1.3 mm).

17. The article of claim 14 wherein said first material has an average pore size of 0.3-10 mm in diameter, and said second material has an average pore size of 0.1-2.0 mm in diameter.

18. The article of claim 14 wherein said first material has an average pore size of 0.5-5.0 mm in diameter, and said second material has an average pore size of 0.3-1.5 mm in diameter.

19. The article of claim 14 wherein said first material has an average pore size of 1.0-2.0 mm in diameter, and said second material has an average pore size of 0.5-1.0 mm in diameter.

20. The article of claim 14 wherein said first material has a basis weight at least as high as said second material, said first material has a basis weight of 25-100 gsm, and said second material has a basis weight of 10-35 gsm.

21. The article of claim 14 wherein said first material has a basis weight of 30-65 gsm, and said second material has a basis weight of 15-30 gsm.

22. The article of claim 14 wherein said first material has a basis weight of 35-50 gsm, and said second material has a basis weight of 20-30 gsm.

23. The article of claim 14 wherein said first material has a basis weight at least as high as said second material.

24. The article of claim 14 wherein said first and second materials are laminated together.

25. The article of claim 14 wherein said first and second materials are contiguous.

26. The article of claim 14 wherein said first and second materials are formed of substantially the same polymer.

27. The article of claim 26 herein said same polymer is polyethylene.

28. The article of claim 14 wherein said pores are generally conical.

29. The article of claim 14 wherein each of said materials is formed of a wettable and substantially non-absorbent thermoplastic polymer.

30. An absorbent article comprising:
(A) a topsheet;
(B) an absorbent core; and
(C) an acquisition/distribution transfer system disposed intermediate said topsheet and said absorbent core;
said system consisting essentially of only one apertured material, said material being a three dimensional and wettable film, said material defining generally conical pores which taper inwardly in a first direction from said absorbent core toward said topsheet and extend appreciably beyond the primary plane in said first direction from said absorbent core toward said topsheet;
said material having an average pore size of 0.3-10 mm in diameter;
said material having a basis weight of 25-100 gsm.

31. An absorbent article comprising:
(A) a topsheet;
(B) an absorbent core; and
(C) a layered acquisition/distribution transfer system disposed intermediate said topsheet and said absorbent core;
said system comprising at least a pair of apertured materials including a first material facing said topsheet and a second material facing said absorbent core, each said material being three dimensional and formed of a wettable and substantially non-absorbent thermoplastic polymer film, each said material defining generally conical pores which taper outwardly in a first direction from said absorbent core toward said topsheet and extend appreciably beyond the respective primary plane of the material in said first direction from absorbent core to said topsheet;
said first material having a larger average pore size than said second material, said first material having an average pore size of 0.3-10 mm in diameter, and said second material having an average pore size of 0.1-2.0 mm in diameter;
said first material having a higher basis weight than said second material, said first material having a basis weight of 25-100 gsm, and said second material having a basis weight of 10-35 gsm;
said first and second materials being contiguous, formed of essentially the same polymer and having a combined thickness of at least 30 mils (0.76 mm).
